(19) United States
(12) Patent Application Publication
(10) Pub. No.: US 2017/0050546 A1
(43) Pub. Date: Feb. 23, 2017

(54) Sleep Restraint System

(71) Applicant: Leonce Estimable, Lantana, FL (US)

(72) Inventor: Leonce Estimable, Lantana, FL (US)

(21) Appl. No.: 14/829,795

(22) Filed: Aug. 19, 2015

(51) Int. Cl. B60N 2/48; A61F 9/04

(52) U.S. Cl. CPC B60N 2/4879; A61F 9/04

(57) ABSTRACT

A sleep restraint system includes a vehicle that has a seat. A brace is provided and the brace may be worn around a neck of a user when the user sits in the seat. Thus, the brace to maintains a head of the user in an upright position when the user sleeps. A head piece is provided and the head piece may be worn on the user's head. Thus, the head piece covers the user's eyes while the user sleeps. The head piece engages the user's jaw thereby facilitating the head piece to retain the user's mouth in a closed position while the user sleeps.

### BACKGROUND OF THE DISCLOSURE

### Field of the Disclosure

The disclosure relates to restraint devices and more particularly pertains to a new restraint device for supporting the head and keeping the jaw closed while a user sleeps.

### SUMMARY OF THE DISCLOSURE

An embodiment of the disclosure meets the needs presented above by generally comprising a vehicle that has a seat. A brace is provided and the brace may be worn around a neck of a user when the user sits in the seat. Thus, the brace to maintains a head of the user in an upright position when the user sleeps. A head piece is provided and the head piece may be worn on the user's head. Thus, the head piece covers the user's eyes while the user sleeps. The head piece engages the user's jaw thereby facilitating the head piece to retain the user's mouth in a closed position while the user sleeps.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a sleep restraint system according to an embodiment of the disclosure.

FIG. 2 is a bottom view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

### DESCRIPTION OF THE PREFERRED EMBODIMENT

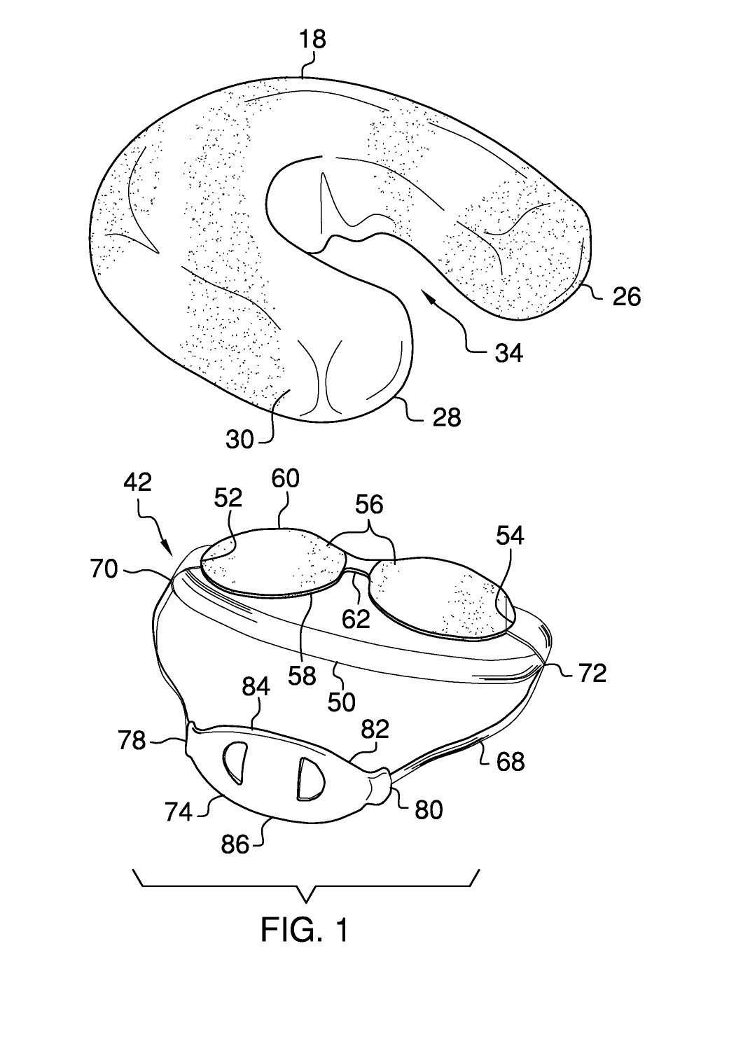

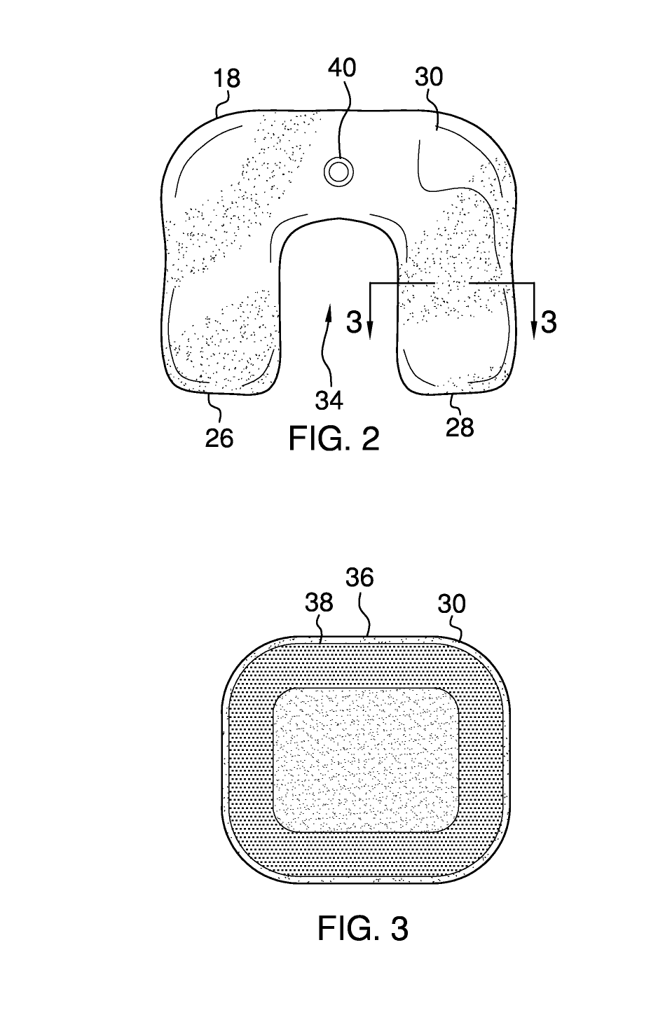

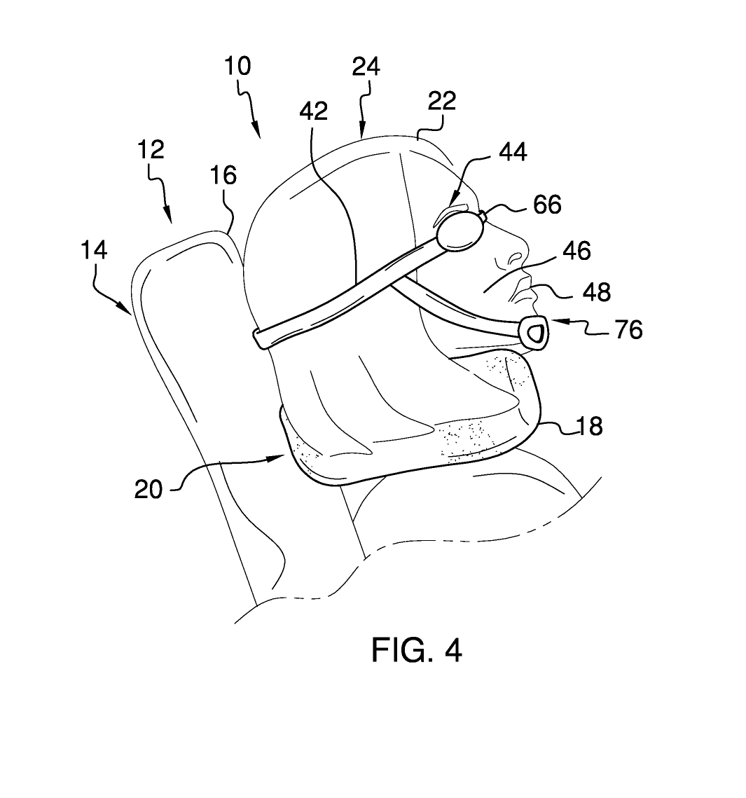

With reference now to the drawings, and in particular to FIGS. 1 through 4 thereof, a new restraint device embodying the principles and concepts of an embodiment of the disclosure and generally designated by the reference numeral 10 will be described.

As best illustrated in FIGS. 1 through 4, the sleep restraint system 10 generally comprises a vehicle 12 that has a seat 14 and the seat 14 has a headrest 16. The vehicle 12 may be an airplane, a bus or other vehicle utilized to transport a plurality of passengers. A brace 18 is provided and the brace 18 may be worn around a neck 20 of a user 22 when the user 22 sits in the seat 14. Thus, the brace 18 maintains a head 24 of the user 22 in an upright position when the user 22 sleeps while the user 22 sits in the seat 14.

The brace 18 has a first end 26, a second end 28 and an outer wall 30 extending between the first end 26 and the second end 28. The outer wall 30 is continuous such that the brace 18 has a substantially cylindrical shape. The brace 18 is substantially hollow and the outer wall 30 has a first surface 32. The outer wall 30 is curved between the first end 26 and the second end 28. The first end 26 is aligned with the second end 28 and the first end 26 is spaced from the second end 28. Thus, a neck space 34 is defined between the first end 26 and the second end 28. The neck space 34 may have the user's neck 20 positioned therein whereby the first surface 32 of the outer wall 30 abuts the user's neck 20.

The outer wall 30 comprises an outer layer 36 and an inner layer 38. The outer layer 36 is comprised of a deformable material. The inner layer 38 is comprised of a resiliently compressible material such as foam rubber or the like. The inner layer 38 has a thickness that is greater than a thickness of the outer layer 36.

A valve 40 is coupled to the brace 18. The valve 40 extends through the outer wall 30 such that the valve 40 is in fluid communication with an interior of the brace 18. The valve 40 may have air selectively urged inwardly therethrough thereby facilitating the brace 18 to be inflated. Thus, the brace 18 supports the user's head 24 when the brace 18 is worn around the user's neck 20. The valve 40 may have air selectively released outwardly therefrom thereby facilitating the brace 18 to be deflated. The brace 18 may be stored when the brace 18 is deflated.

A head piece 42 is provided and the head piece 42 may be worn on the user's head 24 thereby facilitating the head piece 42 to cover the user's eyes 44 while the user 20 sleeps. The head piece 42 may engage the user's jaw 46 thereby facilitating the head piece 42 to retain the user's mouth 48 in a closed position while the user 20 sleeps. The head piece 42 comprises a first strap 50 that has a first end 52 and a second end 54. The first strap 50 may be wrapped around the user's head 24.

A pair of eye patches 56 is provided and each of the eye patches 56 is coupled to an associated one of the first end 52 and the second end 54 of the first strap 50. Each of the eye patches 56 is spaced from each other. Thus, each of the eye patches 56 covers an associated one of the user's eyes 44 when the first strap 50 is worn around the user's head 24. Each of the eye patches 56 has a back surface 58 and a perimeter edge 60. The perimeter edge 60 of each of the eye patches 56 is curved such that each of the eye patches 56 has an ovoid shape. The back surface 58 abuts the user's head 24 when eye patches 56 are positioned to cover the user's eyes 44.

A bridge 62 is coupled to the perimeter edge 60 of each of the eye patches 56 such that the bridge 62 extends between each of the eye patches 56. The bridge 62 retains each of the eye patches 56 a distance apart from each other. The bridge 62 extends across a bridge of the user's nose 66 when the first strap 50 is worn around the user's head 24. Thus, each of the eye patches 56 is positioned to cover each of the user's eyes 44.

A chin strap 68 is provided that has a primary end 70 and a secondary end 72. Each of the primary end 70 and the secondary end 72 is coupled to the first strap 50 and the chin strap 68 extends substantially downwardly from the first strap 50. Thus, the chin strap 68 may extend around the user's jaw 46 when the first strap 50 is worn around the user's head 24. The primary end 70 is positioned proximate the first end 52 of the first strap 50 and the secondary end 72 is positioned proximate the second end 54 of the first strap 50.

The chin strap 68 includes a chin piece 74. The chin piece 74 is centrally positioned on the chin strap 68 such that the chin piece 74 may engage the user's chin 76 when the first strap 50 is worn around the user's head 24. Thus, the chin strap 68 retains the user's mouth 48 in a closed position. The chin piece 74 has a first end 78, a second end 80, a back side 82, a top edge 84 and a bottom edge 86. The chin strap 68 is coupled to each of the first end 78 and the second end 80 of the chin piece 74. Each of the top edge 84 and the bottom edge 86 curves upwardly and downwardly, respectively, between the first end 78 and the second end 80 of the chin piece 74. Thus, the chin piece 74 has an ovoid shape thereby facilitating the chin piece 74 to surround the user's chin 76 when the head piece 42 is worn on the user's head 24.

In use, the brace 18 is worn around the user's neck 20 when the user 22 wishes to sleep. The brace 18 retains the user's head 24 in an upright position such that the user's head 24 does not tilt sideways onto an adjacent passenger in the vehicle 12. The first strap 50 is worn around the user's head 24 and each of the eye patches 56 is positioned over the user's eyes 44. Each of the eye patches 56 prevents the user 22 from being disturbed by light when the user 22 is sleeping. The chins strap 68 is positioned to extend around the user's jaw 46. The chin piece 74 engages the user's chin 88 such that the user's mouth 48 is retained in the closed position. The chin piece 74 prevents the user 22 from drooling while the user 22 sleeps.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sleep restraint system comprising:
a vehicle having a seat;
a brace being configured to be worn around a neck of a user when the user sits in said seat thereby facilitating said brace to maintain a head of the user in an upright position when the user sleeps; and
a head piece being configured to be worn on the user's head thereby facilitating said head piece to cover the user's eyes while the user sleeps, said head piece being configured to engage the user's jaw thereby facilitating said head piece to retain the user's mouth in a closed position while the user sleeps.

2. The assembly according to claim 1, wherein said brace has a first end, a second end and an outer wall extending between said first end and said second end, said brace being substantially hollow, said outer wall having a first surface, said outer wall being curved between said first end and said second end such that said first end is spaced from said second end to define a neck space between said first end and said second end, said neck space being configured to have the user's neck positioned therein whereby said first surface of said outer wall abuts the user's neck, said outer wall comprising a outer layer and a inner layer.

3. The assembly according to claim 1, further comprising a valve being coupled to said brace, said valve extending through said outer wall such that said valve is in fluid communication with an interior of said brace, said valve being configured to have air being selectively urged inwardly therethrough thereby facilitating said brace to be inflated, said valve being configured to have air being selectively released outwardly therefrom thereby facilitating said brace to be deflated.

4. The assembly according to claim 1, wherein said head piece comprises:
a first strap having a first end and a second end, said first strap being configured to be wrapped around the user's head; and
a pair of eye patches, each of said eye patches being coupled to an associated one of said first end and said second end of said first strap.

5. The assembly according to claim 4, wherein each of said eye patches is spaced from each other wherein each of said eye patches is configured to cover an associated one of the user's eyes when said first strap is worn around the user's head, each of said eye patches having a back surface and a perimeter edge, said perimeter edge of each of said eye patches being curved such that each of said eye patches has an ovoid shape.

6. The assembly according to claim 5, further comprising a bridge being coupled to said perimeter edge of each of said eye patches such that said bridge extends between each of said eye patches, said bridge retaining each of said eye patches a distance apart from each other.

7. The assembly according to claim 1, wherein said head piece comprises:
a first strap having a first end and a second end; and
a chin strap having a primary end and a secondary end, each of said primary end and said secondary end being coupled to said first strap such that said chin strap extends substantially downwardly from said first strap wherein said chin strap is configured to extend around the user's jaw when said first strap is worn around the user's head, said primary end being positioned proximate said first end, said secondary end being positioned proximate said second end.

8. The assembly according to claim 7, wherein said chin strap includes a chin piece, said chin piece being centrally positioned on said chin strap wherein said chin piece is configured to engage the user's chin when said first strap is worn around the user's head thereby facilitating said chin strap to retain the user's mouth in a closed position.

9. The assembly according to claim 8, wherein said chin piece has a first end, a second end, a back side, a top edge and a bottom edge, said chin strap being coupled to each of said first end and said second end of said chin piece, each of said top edge and said bottom edge respectively curving upwardly and downwardly between said first end and said second end of said chin piece such that said chin piece has an ovoid shape wherein said chin piece is configured to surround the user's chin when said head piece is worn on the user's head.

10. A sleep restraint system comprising:
a vehicle having a seat, said seat having a headrest;
a brace being configured to be worn around a neck of a user when the user sits in said seat thereby facilitating said brace to maintain a head of the user in an upright position when the user sleeps, said brace having a first end, a second end and a outer wall extending between said first end and said second end, said brace being substantially hollow, said outer wall having a first surface, said outer wall being curved between said first end and said second end such that said first end is spaced from said second end to define a neck space between said first end and said second end, said neck space being configured to have the user's neck positioned therein whereby said first surface of said outer wall abuts the user's neck, said outer wall comprising a outer layer and a inner layer;
a valve being coupled to said brace, said valve extending through said outer wall such that said valve is in fluid communication with an interior of said brace, said valve being configured to have air being selectively urged inwardly therethrough thereby facilitating said brace to be inflated, said valve being configured to have air being selectively released outwardly therefrom thereby facilitating said brace to be deflated; and
a head piece being configured to be worn on the user's head thereby facilitating said head piece to cover the user's eyes while the user sleeps, said head piece being configured to engage the user's jaw thereby facilitating said head piece to retain the user's mouth in a closed position while the user sleeps, said head piece comprising:
a first strap having a first end and a second end, said first strap being configured to be wrapped around the user's head,
a pair of eye patches, each of said eye patches being coupled to an associated one of said first end and said second end of said first strap, each of said eye patches being spaced from each other wherein each of said eye patches is configured to cover an associated one of the user's eyes when said first strap is worn around the user's head, each of said eye patches having a back surface and a perimeter edge, said perimeter edge of each of said eye patches being curved such that each of said eye patches has an ovoid shape,
a bridge being coupled to said perimeter edge of each of said eye patches such that said bridge extends between each of said eye patches, said bridge retaining each of said eye patches a distance apart from each other,
a chin strap having a primary end and a secondary end, each of said primary end and said secondary end being coupled to said first strap such that said chin strap extends substantially downwardly from said first strap wherein said chin strap is configured to extend around the user's jaw when said first strap is worn around the user's head, said primary end being positioned proximate said first end, said secondary end being positioned proximate said second end, said chin strap including a chin piece, said chin piece being centrally positioned on said chin strap wherein said chin piece is configured to engage the user's chin when said first strap is worn around the user's head thereby facilitating said chin strap to retain the user's mouth in a closed position, said chin piece having a first end, a second end, a back side, a top edge and a bottom edge, said chin strap being coupled to each of said first end and said second end of said chin piece, each of said top edge and said bottom edge respectively curving upwardly and downwardly between said first end and said second end of said chin piece such that said chin piece has an ovoid shape wherein said chin piece is configured to surround the user's chin when said head piece is worn on the user's head.
